(19) United States
(12) Patent Application Publication
(10) Pub. No.: US 2026/0232240 A1
(43) Pub. Date: Aug. 13, 2026

(54) METHOD FOR DETERMINING AN EMOTIONAL STATE OF A SUBJECT AND ASSOCIATED DEVICE

(71) Applicant: EMOBOT, Paris (FR)

(72) Inventor: Tanel PETELOT, Paris (FR)

(73) Assignee: EMOBOT, Paris (FR)

(21) Appl. No.: 19/143,951

(22) PCT Filed: Jan. 4, 2024
(86) PCT No.: PCT/FR2024/050005

§ 371 Date: May 5, 2026

(30) Foreign Application Priority Data
Jan. 6, 2023 (FR) FR2300155

(51) Int. Cl. A61B 5/16; A61B 5/00

(52) U.S. Cl. CPC A61B 5/165; A61B 5/7246; A61B 2503/08

(57) ABSTRACT

A computer-implemented method for determining, at an evaluation time (te), an emotional state S(te) of a person (P) immersed in an environment (E) for the purpose of monitoring an emotional state of said person (P). An associated determination system and a computer program product.

### FIELD

The present invention relates to the determination of an emotional state of a person for monitoring the state of health thereof.

The present invention relates to a computer-implemented method for determining, at an evaluation time, an emotional state of a subject immersed in an environment for the purpose of monitoring an emotional behaviour of this subject and of predicting the state of health thereof.

### BACKGROUND

Depressive disorders or mental health disorders are common illnesses that affect a large part of the global population. Mental health disorders are treatable, but very few patients receive treatment. Generally, illnesses related to mental health disorders do not improve on their own, and if left untreated, they can worsen and cause other serious problems. Often, depressed people are not aware of the presence of symptoms of depression, such as apathy, when they are affected.

Examples of current ways to monitor the emotional state of people are the following.

For example, monitoring the emotional state of a person placed in a Residential Care Facility for the Elderly (RCFE) or residing at home may be carried out on the basis of information being passed on to a doctor (psychiatrist, gerontologist, geriatrist). The information is entered into an IT system by a care team and passed on to the doctor. The disadvantage of this method is the subjectivity of the information entered by the care team and the impact of this subjectivity on the clarity of the information. This makes it difficult for the doctor receiving the information to monitor the emotional behaviour of the person based on this information, in order to prescribe treatment to the person.

In another example, questionnaires can be performed through interviews conducted by a psychologist. This type of questionnaire usually comprises a small amount of information (for example, when it is a questionnaire related to the Geriatric Depression Scale (GDS), consisting of a limited number of questions that can be reduced to four questions). Furthermore, questionnaires are generally completed in the best case every two weeks.

In yet another example, the doctor (prescribing doctor or psychiatrist) may perform a clinical interview during which they ask the subject questions to understand the emotional state thereof. This type of clinical interview is time-consuming and, in the best case, is conducted at a rate of one interview per month.

The invention overcomes these drawbacks.

### SUMMARY

In this context, the invention proposes a solution aiming to monitor the emotional state of a subject immersed in an environment in order to predict the state of health thereof (i.e., emotional state).

A first aspect of the invention relates to a computer-implemented method for determining, at an evaluation time, an emotional state of a person immersed in an environment for the purpose of monitoring an emotional state of said person, said method comprising the following steps of:
receiving, during all or part of a time period preceding the evaluation time, at least one first signal acquired by at least one sensor, said first signal being representative of a first time sequence of behavioural and/or physiological attributes of the person during said time period;
receiving, during all or part of said time period, at least one second signal acquired by at least one environment sensor, said second signal being representative of a second time sequence of states of the environment during said time period;
determining, based on the at least one first signal, a third time sequence of emotional signatures of the subject during said time period, said third sequence forming a first set of emotional signatures of the subject, each of the emotional signatures of the person of the third sequence being represented by a position in a space representative of the emotions;
determining, based on the at least one second signal, a fourth time sequence of signatures of the environment during said time period, said fourth sequence forming a second set of signatures of the environment, each of the signatures of the environment being represented by a position in said space representative of the emotions;
determining a degree of similarity between the first set of emotional signatures of the subject and the second set of signatures of the environment;
determining the emotional state of the person at the evaluation time based on the degree of similarity determined.

Thus, the determination method presented here makes it possible, by comparing between the emotional signatures of a person over a given period and the signatures of the environment over the same given period, to estimate an emotional contagion of the environment of the person to the latter, and to estimate the reactivity of the person to the environment thereof. For example, this makes it possible to estimate whether the person has apathy.

In some embodiments, determining the degree of similarity between the first set of emotional signatures of the person and the second set of signatures of the environment comprises calculating a distance in said representative space between the first set of emotional signatures of the person and the second set of signatures of the environment.

In some embodiments, the distance is calculated by means of a cosine similarity between a first vector representative of the first set of emotional signatures of the person and the second set of signatures of the environment.

In some embodiments, the calculation of the distance comprises a weighting dependent on the positions of the emotional signatures of the subject of the first set and of the signatures of the environment of the second set in said representative space.

In some embodiments, the calculation of said distance comprises a time-dependent weighting corresponding to the emotional signatures of the person in the third time sequence and the signatures of the environment in the fourth time sequence.

In some embodiments, the calculation of said distance is carried out between the emotional signatures of the person of a first sub-sequence of the third time sequence and the signatures of the environment of a second sub-sequence of the fourth time sequence, said first sub-sequence and second sub-sequence corresponding to a time sub-sequence of said time period.

In some embodiments:
the at least one first signal is acquired by the at least one sensor chosen from: a camera, a webcam, a three-dimensional camera, a microphone, an inertial unit, a physiological sensor, a headset dedicated to electroencephalographic measurements,
the method is configured to extract from the at least one first signal the behavioural and/or physiological attributes in the first time sequence so as to obtain values representative of at least one of the following attributes from: a gesture of the person, a movement of the person, a facial expression of the person, a movement of the eyes of the person, physiological parameters of the person, interaction with an electronic device.

In some embodiments:
the at least one second signal is acquired by the at least one environment sensor chosen from: a camera, a webcam, a three-dimensional camera, a microphone, a means of accessing a remote server storing information relating to one or more audiovisual, visual, or audio content broadcast during all or part of the time period, an electromagnetic detector,
and in that the method is configured to extract from the at least second signal the state of the environment, so as to obtain values representative of a state chosen from: an amount of movement in audiovisual or visual content, broadcast during all or part of said time period, a sound level and/or a frequency spectrum of an audiovisual or audio content broadcast during all or part of said time period, a sound level and/or a sound frequency spectrum in a neighbourhood close to the subject, an electromagnetic signal representative of a variation of an electromagnetic signature of a neighbourhood close to the subject.

A second aspect of the invention relates to a system for determining, at an evaluation time, an emotional state of a person immersed in an environment in order to monitor a state of health of said person, comprising at least one sensor, at least one environment sensor, and a programmable device, said programmable device being suitable for:
receiving, during all or part of a time period preceding the evaluation time, at least one first signal acquired by the at least one sensor, said first signal being representative of a first time sequence of behavioural and/or physiological attributes of the subject during said time period;
receiving, during all or part of said time period, at least one second signal acquired by the at least one environment sensor, said second signal being representative of a second time sequence of a state of the environment during said time period;
determining, based on the at least one first signal, a third time sequence of emotional signatures of the person during said time period, said third sequence forming a first set of emotional signatures of the person, each of the emotional signatures of the person of the third sequence being represented by a position in a space representative of a panel of emotions;
determining, based on the at least one second current signal, a fourth time sequence of signatures of the environment during said time period, said fourth sequence forming a second set of signatures of the environment, each of the signatures of the environment being represented by a position in said representative space;
determining a degree of similarity between the first set of emotional signatures of the subject and the second set of signatures of the environment,
determining the emotional state of the person at the evaluation time based on the degree of similarity determined.

A third aspect of the invention relates to a computer program including instructions for implementing the following steps of a method for determining, at an evaluation time, an emotional state of a person immersed in an environment for the purpose of monitoring a state of health of said person during the execution of the program by a processor of a programmable device:
receiving, during all or part of a time period preceding the evaluation time, at least one first signal acquired by at least one sensor, said first signal being representative of a first time sequence of behavioural and/or physiological attributes of the subject during said time period;
receiving, during all or part of said time period, at least one second signal acquired by at least one environment sensor, said second signal being representative of a second time sequence of a state of the environment during said time period;
determining, based on the at least one first signal, a third time sequence of emotional signatures of the person during said time period, said third sequence forming a first set of emotional signatures of the person, each of the emotional signatures of the person of the third sequence being represented by a position in a space representative of a panel of emotions;
determining, based on the at least one second current signal, a fourth time sequence of signatures of the environment during said time period, said fourth sequence forming a second set of signatures of the environment;
determining a degree of similarity between the first set of emotional signatures of the subject and the second set of signatures of the environment;
determining the emotional cognitive state of the person at the evaluation time based on the degree of similarity determined.

The present disclosure also relates to a non-transitory computer-readable recording medium comprising instructions which, when they are executed by a computer, cause it to implement a method according to any one of the embodiments.

Such a non-transitory computer-readable recording medium may be, without limitation, an electronic, magnetic, optical, electromagnetic, infrared or semiconductive device, or any suitable combination of the above. It should be noted that the following examples, although more specific, are only an illustrative and non-exhaustive list, easily assessed by a person having ordinary knowledge of the art: a laptop floppy disk, a hard disk, an ROM, an EPROM (Erasable Programmable ROM) or a Flash memory, a portable CD-ROM (Compact-Disc ROM).

### Definitions

In the present invention, the terms below are defined as follows:
The expression “Emotional cognitive state of a person” refers to a state of mind such as a sensation, an emotion, a feeling, a mood resulting from a set of mental phenomena that influence both the own state of mind, attitude, world view, thinking, and behaviour in the world, of the person.
The expression “Negative state” of a person refers to a state of mind that generates a mental suffering of the person.
The expression “Emotional signature” refers to a representation of an emotional state in a space representative of the emotions.
The expression “Environment of a person at a given time” refers, within the scope of the present description, to the set of physical elements with which the person interacts or which are likely to induce a reaction of the person. For example, the environment may include other people interacting with the analysed person, a robot such as a humanoid, a connected speaker, or a chatbot, a sound and/or visual environment, such as a telephone, a laptop, a television or more broadly information sources broadcasting signals and perceptible to the person at the given time.
The expression “state of the environment” refers, within the scope of the present description, to a characterisation of sensory stimuli present in the environment that are likely to change the emotional state of a person.
The expression “signature of the environment” refers, within the scope of the present description, to a representation in a space representative of the emotions of a state that the environment would be likely to diffuse and therefore be perceived by a person located in this environment.
“Apathy” is defined as an indolence or indifference of a person, pushed to complete insensitivity.
“Emotional tone” refers, within the scope of the present description, to all of the emotional states through which the person P goes during a given period. Thus, the emotional tone makes it possible to infer the dynamism, vigour and energy evaluated through the amplitude of the emotions produced by a subject.
The term “heat map” refers to a graphical representation of an accumulation of emotions, in the form of an image represented by a two-dimensional array of pixels, each pixel representing an emotion. The intensity of each pixel in the heat map is related to the number of times the emotion associated with the pixel has been identified over the period in which the accumulation is calculated.

### BRIEF DESCRIPTION OF THE FIGURES

FIG. 1 is an example of steps implemented for performing the method for determining an emotional state of a subject, according to one or more embodiments.

FIG. 2 represents an example of a time sequence of emotional signatures of a person represented in a space representative of the emotions.

FIG. 3 shows an example of a heat map of emotions translating an emotional tone of a person into a space representative of the emotions.

FIG. 4 represents an example of a time sequence of signatures of an environment represented in a space representative of the emotions.

FIGS. 5a and 5b show two heat maps of emotions of a healthy person at two different evaluation times within a given evaluation period. FIG. 5c represents a heat map of emotions of the same person determined at the end of the given evaluation period.

FIGS. 6a, 6b and 6c show three heat maps of emotions of a person suffering from depression, at three different evaluation times within a given evaluation period. FIG. 6d represents a heat map of emotions of the same person, determined at the end of the given evaluation period.

FIG. 7 shows, an emotion heat map of a person determined during a given evaluation period, to the left, and an emotion heat map produced by an environment of the person, during the given evaluation period, to the right.

FIG. 8 is a schematic diagram showing some of the components of an example of a determination system configured to implement the method for determining an emotional state of a subject.

FIG. 9 is a curve representative of the change in the emotional tone of a person entering an RCFE, wherein the emotional tone is determined by the determination method described here.

### DETAILED DESCRIPTION

A first aspect of the invention relates to a computer-implemented method 100 for determining, at an evaluation time tₑ, an emotional state S(tₑ) of a person P immersed in an environment E.

Indeed, by determining and monitoring the change in the emotional state S(t) of the person P between a time t₁ preceding the evaluation time tₑ and the evaluation time tₑ, it is desired to be able to detect negative states, or to detect a change in the emotional state of the person towards a negative state, in order to monitor the state of health thereof. A particular application is the detection of warning signs of depression in a person. In this application, it is advantageous to determine the emotional cognitive state of the person at various times tₖ, k being an integer between 1 and K.

The method 100 is for example implemented by a system for determining 1 an emotional state of a person P immersed in an environment E. As will be explained later, the method 100 uses a plurality of signals captured between an initial time t₁ and the evaluation time tₑ, to determine the emotional state of the person P at the evaluation time tₑ.

Advantageously, the determination system 1 comprises a plurality of sensors CPᵢ intended to monitor the person P, a plurality of environment sensors CEⱼ and a programmable device, i and j being integers between 1 and I, and 1 and J respectively.

Programmable device means any programmable information processing system such as a processor, or a company computer, a personal computer (PC), a smartphone or a touch tablet.

The plurality of sensors CPᵢ is intended to acquire a plurality of first signals Spᵢ(t) during the time period [t₁,tₑ]. Each of the first signals Spᵢ(t) is representative of a first time sequence relating to a behavioural and/or physiological attribute of the person P. Behavioural attribute of a person means an attribute relating to the behaviour thereof, such as the movements of the body thereof or the facial expression thereof. A physiological attribute of a person means an attribute related to the physiology thereof, such as the heart rate thereof, the brain activity thereof, the respiratory rate thereof. The first signals Spᵢ(t) may be discrete or continuous. Moreover, they are either scattered over the time period [t₁,tₑ] or recorded according to an ordered calendar over the time period [t₁,tₑ]. For example, the first signals Spᵢ(t) may be recorded first once a week, then once a day, then every second for one hour each day.

The plurality of environment sensors CEᵢ is intended to acquire a plurality of second signals Seⱼ(t) during the time period [tᵢ,tₑ]. Each of the second signals Seⱼ(t) is representative of a second time sequence of states of the environment E. The second signals Seⱼ(t) may be discrete or continuous. Moreover, they are either scattered over the time period [t₁,tₑ] or recorded according to an ordered calendar over the time period [t₁,tₑ]. For example, the second signals Seⱼ(t) may be recorded first once a week, then once a day, then every second for one hour each day.

For example, the plurality of sensors CPᵢ comprises a sensor C₁ consisting of a camera, or a webcam, or a three-dimensional camera filming at least one part of the body of the person P. The at least one part of the body comprises the face of the person P. In this case, the first signal Sp₁(t) is representative of a change in the facial expression of the person P, the movement of the eyes thereof or the movements of the head thereof over time.

Typically, the movement of reference points on the images captured by the camera, such as points located on the contour of the mouth of the person P, or on the eyebrows thereof, is analysed in order to profile the emotion of the person. This may consist, for example, in identifying a smile on the face of the person P, or a frowning of eyebrows.

The detection of points representative of an expression (those characterising the contour of the eyes and of the mouth, for example) can be performed by Machine Learning algorithms such as Random Forest or Deep Learning algorithms such as convolutional neural networks. These tools can also be used to recognise the facial expression from six expressions such as Ekman's expressions, without going through the analysis of points representative of the face, but directly from the pixels of the image representing the face. These techniques have become commonplace and are built into smartphones to automatically trigger taking a shot when the person being photographed smiles.

For example, the plurality of sensors CPᵢ comprises a sensor C₂ consisting of a microphone that can record the voice of the person P. In this case, the first signal Sp₂(t) is representative of a change in the tone and/or amplitude of the voice of the person P. Just as a classifier can analyse facial expression, it is possible to rely on characteristics of the audio signal Sp₂(t) such as cepsters or formants to identify the emotion contained in the voice by means of classifiers such as auto-encoders or neural networks

For example, the plurality of sensors CPᵢ comprises a sensor C₃ consisting of an inertial unit worn by the person P, for example embedded in a connected object worn by the person P such as a connected watch, and that can record the position thereof and the movements thereof. In this case, the first signal Sp₃(t) is representative of a change in the gesture of the person P and the movements of the body thereof. It is possible to deduce from the first signal Sp₃(t) an estimation of the posture (the skeleton characterised by a set of points generally corresponding to the joints) of the person. Analysing emotions from the posture then involves analysing the movement of the person over time to deduce therefrom the emotion. Tools from the deep learning field such as LSTM can be used, as well as other tools from the machine learning field dedicated to the recognition of time forms.

For example, the plurality of sensors CPᵢ comprises a physiological sensor that can detect the physiological signals of the person P. For example, the physiological sensor may be a sweat or heart rate sensor. For example, a high heart rate will make it possible to indicate a state of stress or extreme agitation of the person P. Commercial sensors can be used (such as Biopac MP150, Empatica E4, etc.), the signals of which can be analysed by machine learning.

For example, again, the plurality of sensors CPᵢ comprises a headset dedicated to electroencephalographic measurements, in order to detect the brain activity of the person P, such as the concentration level thereof or the stress state thereof.

For example, the plurality of sensors CPᵢ comprises a sensor capable of measuring the presence of a person other than the person P in a room, such as a motion sensor, a magnetic, electromagnetic fingerprint detector, an infrared detector.

For example, the plurality of environment sensors CPi comprises an electromagnetic detector such as a fall detection system using RADAR technology, such as the product Morphee+™. Such a sensor makes it possible to analyse the movement of the person in the environment thereof. As with other conventional motion sensors, such a sensor therefore makes it possible to analyse the movement of the person and associate it with emotions. For example, a rapid movement (associated with a positive emotion) when the person enters the bedroom thereof will easily be differentiated from a slow movement that will reflect a retreat, a lack of vitality very characteristic of depression.

For example, the plurality of environment sensors CEⱼ comprises an environment sensor CE₁ consisting of a camera, a webcam, a three-dimensional camera that can film the place where the person P is located, for example, a room. In this case, the second signal Se₁(t) may be representative of the presence or the absence of other people in the room.

For example, the plurality of environment sensors CEⱼ comprises an environment sensor CE₂ consisting of a microphone. In this case, the second signal Se₂(t) can be representative of the sounds emitted in the environment where the person P is located. The difficulty then consists in separating the audio sources from the environment to identify the origin thereof. For example, recurring variable auto-encoders used in the deep learning field make it possible to separate voice audio sources. It is then possible to identify the voice of the person P and subsequently focus, for example, on another voice existing in the environment if, for example, another person P′ is in conversation with the person P. The audio emotion recognition tools are then applied to recognise the emotion contained in the voice of the person P′ that constitutes the emotion produced in the environment of the person P.

For example, the plurality of environment sensors CEⱼ comprises an environment sensor CE₃ consisting of a means of accessing a remote server storing information relating to one or more audiovisual, visual, or audio content broadcast during all or part of the time period [t₁,tₑ]. Such an access means may for example be an Internet connection making it possible to access the description of an audiovisual programme being played by the person P (comedy, drama, etc.). For example, if the current program is a comedy, the emotional content of the environment will present joyful and positive states. Conversely, if the programme is a drama, the emotional content of the environment will present anxious and stressful states.

In general, some of the environment sensors CEⱼ may coincide with some of the sensors Cᵢ.

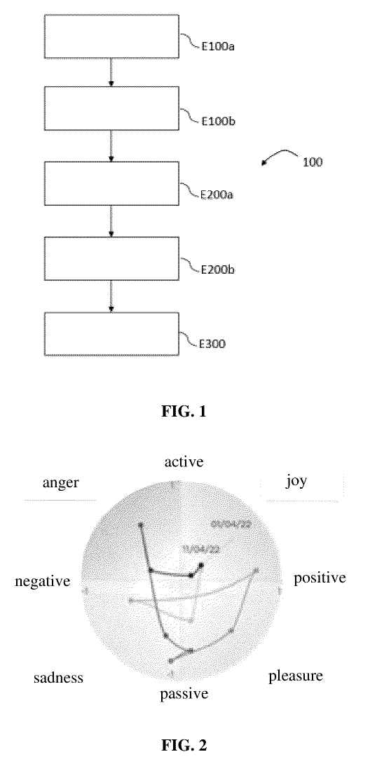

The method 100 for determining an emotional state of the person P, at the evaluation time tₑ, will now be described. FIG. 1 illustrates the main steps performed during the implementation of the determination method 100.

In a reception step relating to the person P, E100a, all of the first signals Spᵢ(t) are received by the various sensors CPᵢ over the time period [t₁,tₑ].

In a reception step relating to the environment E, E100b, all of the second signals Sej(t) are received by the various environment sensors CEⱼ during the time period [t₁,tₑ].

Typically, steps E100a and E100b are concurrent. Indeed, both take place during the time period [t₁,tₑ].

In a determination step E200a relating to the person P and subsequent to the steps E100a and E100b, a third time sequence of emotional signatures of the person P is determined based on the set of the first signals Spᵢ(t). All of the emotional signatures of the third time sequence form a first set. Each emotional signature is represented by a position in a space representative of the emotions R. For example, a first signal Sp₁(t) corresponding to images of the face of the person P, and therefore comprising representations of the expressions of the face thereof, will be analysed to determine the corresponding emotional signatures.

For example, five sensors CPᵢ are considered, such as a webcam recording images captured at 25 Hz, a microphone recording a sound sampled at 48 KHz, an infrared sensor recording an infrared image sampled at 50 Hz, a wristband recording at a sampling frequency of 100 Hz sweating at the wrist and heart rhythm at a sampling frequency of 10 KHz. Each of the recorded signals is pre-processed appropriately as previously exemplified, namely: by convolutional neural networks to process the images, formants to analyse sound, derivatives for sweating and heart rhythm. Individually, each pre-processing result is used to perform the regression operation which consists, for each sensor CPᵢ, in analysing the pre-processing result to estimate the emotion experienced by the person.

By way of example of embodiment, a network of artificial neurons which will have as input the images of the face of the person will produce as output the coordinates in the Valence Arousal space of the emotion contained in these images. This network will have been previously trained in supervised or unsupervised mode, to produce such an output from the face images. Similarly, the speech produced by the person may be processed to provide a spectrogram whose energy maxima will represent the formants. All of these formants during the period considered [t₁,tₑ] may be projected into a reduced space (or variety) making it possible to estimate the emotion contained in the voice signal.

More generally, the signals recorded by each of the sensors CPᵢ are used to estimate the emotion experienced by the person P by means of signal processing tools such as for example machine learning or deep learning.

Thus, five emotions are estimated from the five pre-processed signals and the estimated emotion associated with each of the pre-processed signals. The fusion of the five emotions is achieved, for example, by an average to identify the final emotion of the person in the Valence Arousal space.

The space representative of the emotions R may be, by way of non-limiting examples:
a discrete space of emotions, for example: a binary measure of positive/neutral, negative/positive or positive/negative emotions, or a discrete set of n emotions, such as Ekman's six emotions,
a continuous space of emotions: for example, the valence/arousal space, a latent space characterised by an analysis in main components, by a generative adversarial network (GAN), or by an auto-encoder.

FIG. 2 shows an example of representation in the valence/arousal space of a third time sequence of emotional signatures of a person P. The valence corresponds to the negative (to the left) or positive (to the right) aspect of the emotional signature. Arousal is the passive (downward) or active (upward) aspect of the emotion. The space around the origin (intersection of the horizontal and vertical axes) is the seat of neutral emotions. The further away from the origin to the periphery, the greater the intensity of the emotion. Each black sampling point (represented by a filled circle) represents an emotional signature at a time tₖ. The set of chronologically arranged emotional signatures forms a trajectory.

A representation of the accumulation of points of the trajectory defined by the set of sampling points consists of an emotion heat map that can be built based on the set of emotional signatures of the person P determined during the time period [t₁,tₑ]. During this time period, a histogram is produced of all of the emotional signatures determined at various times during the time period [t₁,tₑ]. The level of occurrence of the emotional signatures is visualised in the form of a colour level of the pixels corresponding to each emotional signature in the two-dimensional matrix representing the valence/arousal space.

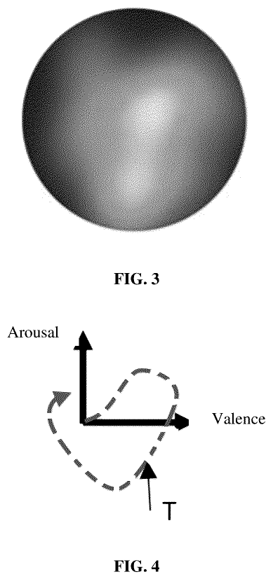

FIG. 3 gives an example of such a heat map, in the valency/arousal space. Black pixels correspond to a low frequency, or low accumulation of the corresponding emotional signature and light pixels correspond to a high frequency, or high accumulation of the corresponding emotional signature. In other words, light pixels correspond to a strong accumulation of the same emotional signature.

In a determination step E200b relating to the environment E, subsequent to the steps E100a and E100b, a fourth sequence of signatures of the environment E is determined based on the set of second signals Seⱼ(t). All of the signatures of the environment of the fourth time sequence form a second set. Each signature of the environment is represented by a position in the space representative of the emotions R, where the emotional signatures of the person P are represented during the time period [t₁,tₑ].

FIG. 4 shows an example of representation in the valence/arousal space of a fourth time sequence of signatures of an environment E. All of the chronologically ordered signatures form a trajectory T (represented as a dashed line).

Similar to the emotional signatures of the person P, it is possible to build a heat map of states of the environment E.

As will be described hereinafter, the determination method 100 presented here makes it possible to detect warning signs of depression in a person.

One of the aspects of the invention is indeed based on the fact that if the person P is healthy, they will be reactive to the environment E. In other words, the emotional state thereof will be close to the state of the environment E. This proximity can be called “emotional contagion”. On the contrary, if the state of health of the person P deteriorates, they will become less and less reactive to the environment E. In other words, the emotional state thereof will move away from the state of the environment E.

An interesting factor to observe is then the emotional tone of a person P. Emotional tone means all of the emotional states that the person P goes through during a given period. A healthy subject passes through all emotional states. On the other hand, the dynamics of the emotions of a person beginning to fall into depression gradually decrease. In other words, the emotional tone of this person narrows around a zone of neutral emotions. In the valence/arousal space, the neutral emotion zone is located around the intersection of the valence axis and of the arousal axis, that is to say in the centre of the space.

The emotional tone can be estimated based on the area in the valence/arousal space of the light pixel area covering the space. The wider this zone, the more satisfying the emotional tone of the person. The narrower this zone, the worse the emotional tone of the person.

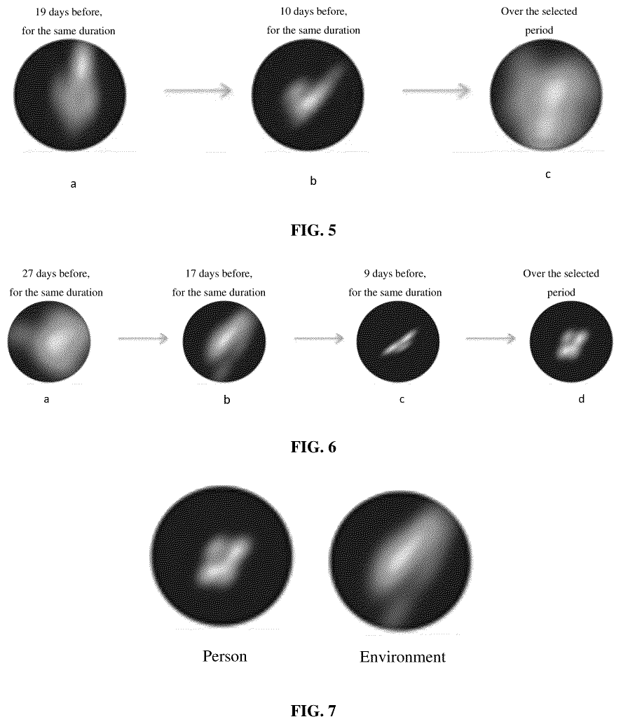

FIGS. 5a and 5b show two emotional heat maps, in the valence/arousal space, of a person with an intermediate emotional tone at two successive times, respectively 19 days before the evaluation time tₑ and 10 days before the evaluation time tₑ. FIG. 5c shows the emotion heat map of the same person over the time period [t₁,tₑ]. It can be observed that the light pixels of FIG. 5c cover a major part of the valence/arousal space. The emotional tone associated with FIG. 5c is good.

FIGS. 6a, 6b and 6c show three heat maps of emotions of a person with depression, at different successive times, respectively 27 days before the evaluation time tₑ, 17 days before the evaluation time tₑ and 9 days before the evaluation time tₑ. FIG. 6d shows the heat map of emotions of the same person over the time period [t₁,tₑ]. It can be observed in FIG. 6a that the emotional tone of the person is good, in other words, that the latter leaves with a good state of health. It can be observed in FIG. 6b that at this evaluation time, the emotional tone of the person is intermediate, compared to the observation in FIG. 6a. It can be observed that the light pixels of FIGS. 6c and 6d only cover a reduced portion, centred around the zone of neutral emotions, of the valence/arousal space, which reflects a weak emotional tone.

Thus, one of the advantageous applications of the method 100 for determining an emotional state described here is the detection of a symptom of developing apathy of the person P. Apathy is defined as indolence or indifference of someone, pushed to complete insensitivity. Apathy is a precursor symptom of depression. The determination method 100 described here thus makes it possible, as already mentioned, to detect warning signs of this pathology.

Thus, in a comparison step E300 subsequent to the steps E200a and E200b, the determination system 1 determines a degree of similarity between the first set and the second set. As a reminder, the first set is the set of emotional signatures of the third time sequence determined during step E200a and the second set is the set of signatures of the environment of the fourth time sequence determined during step E200b.

For example, during the comparison step E300, a heat map of emotions M₁ of the person P and a heat map of states M₂ of the environment E thereof can be compared, at the evaluation time tₑ. The degree of similarity can then be a distance between the heat map of emotions M₁ and the heat map of states M₂. The distance is compared with a threshold value.

FIG. 7 shows to the left a heat map of emotions M₁ of the person P and to the right a heat map of states M₂ of the environment E thereof corresponding to the accumulation of measurements during a period Pe. The emotional signatures corresponding to the map M₁ come from the analysis of the emotions expressed on the face of the person P. The signatures of the environment corresponding to the map M₂ come from the analysis of the sound content of a television programme viewed by the person P.

In FIG. 7, it can be observed that the emotional content that the television programme is likely to convey to the subject is emotionally varied, ranging from passive emotions at the bottom to positive and active manifestations at the top right of the map M₂. This emotional content can be quantified by the area occupied by the light pixels on the heat map. The emotions expressed by the person P exposed to the audio-visual stimuli of the television programme remain concentrated around the region associated with neutral emotions, as seen on the map M₁. A preliminary conclusion may be that the person P is not in tune with the television programme.

If the distance is less than the threshold value, the heat map of emotions M₁ and the heat map of states M₂ are considered to be close. In other words, the method 100 determines that there has been an emotional contagion from the environment E to the person P.

If the distance is greater than the threshold value, the method 100 determines that there is no longer emotional contagion from the environment E to the person P. In particular, it is deduced therefrom that the person P has become apathetic.

The distance between the heat map of emotions M₁ and the heat map of states M₂ is advantageously a distance defined in a vector space (such as the Euclidian distance, the Mahalanobis distance).

Alternatively, the degree of similarity determined in step E300 is a measure of similarity determined by an artificial neural network, or other techniques known in the field of image processing or machine learning.

Advantageously, in the case where the degree of similarity determined in step E300 is a distance calculated between a heat map of emotions M₁ of the person P and a heat map of states M₂ of the environment E thereof, this degree of similarity may be a function of a plurality of parameters. It is assumed that the arousal valence space is a set of N pixels, where each pixel corresponds to an emotion. The heat map of emotions M₁ and the heat map of states M₂ each consist of a set of N numerical values. Each numerical value of the map M₁ or of the map M₂ is representative of the occurrence of the emotion associated with the corresponding pixel during the period [t₁,tₑ].

For example, a first PixelSet parameter corresponds to all of the data used to calculate the distance d. For example, the PixelSet parameter corresponds to all of the N pixels of the valence/arousal space.

For example, a second Filter parameter is a set of coefficients each assigned to one pixel of the set of N pixels of the valence/arousal space. Each coefficient may be chosen so as to favour the importance of the emotion corresponding to the pixel to which the coefficient is assigned in the calculation of the distance.

The second Filter parameter can for example be represented by the binary image of a black disk (black=0) with a radius of 0.5 (if the Valence Arousal space is represented between −1 and 1 on each axis) on a white background (white=1), this disk being centred in [0.0] in the Valence Arousal space. Taking this into account will make it possible to consider only emotions with a certain amplitude: between the edges of this disc of 0.5 radius and the Valence Arousal disc of radius 1. Thus, the neutral emotions will not be taken into account in the calculation of the distance, but only the significant emotions close to the periphery.

For example, a third parameter TimeFilter is a set of coefficients assigned to the measurement times tₚ of the first signals Spᵢ(t) and the second signals Ssⱼ(t) during the period [t₁,tₑ] in order to favour the importance of certain measurement times tₚ in the calculation of the distance. For example, significant coefficients can be assigned to the measurement times tₚ closest to the evaluation time tₑ, to promote the importance of the last emotional signatures of the person P and the last signatures of the environment E thereof.

For example, a fourth Latency parameter makes it possible to take into account the latency time that may exist between the occurrence of a state of the environment and the effect of this occurrence on the emotional state of the person P. For example, the Latency parameter is a vector comprising the values of a discretised Heaviside function whose transition between the value 0 and the value 1 is a function of the latency time considered.

In an advantageous embodiment, the degree of similarity determined in step E300 is a cosine similarity between a first vector formed of the N digital values corresponding to the heat map of emotions M₁ and a second vector formed of the N digital values corresponding to the heat map of states M₂ of the environment E. The cosine similarity Sim between two vectors A and B is defined by the scalar product of these two vectors divided by the product of the norm thereof:

$$Sim = A . B / ( ‖ A ‖ * ‖ B ‖ ) .$$

In this case, the degree of similarity is between 1 and −1. When the degree of similarity is close to 1, there is a significant consistency between the first vector and the second vector, and the latter are considered close, while when the degree of similarity is close to −1, the first vector and the second vector are considered far away.

Another interesting factor to observe is the synchronisation of emotional behaviour breaks between the change in the emotional states of the person P and the change in the states of the environment E thereof. For example, if the environment emits a particular emotion Ee1(t) at a time t and a second thereafter a particular emotion Ep1(t) from the person P is captured, even if the emotion Ep1(t) is different from the state Ee1(t) of the environment, then the emotional changes between the person and the environment thereof are synchronised which means that the person is sensitive to the emotional character of the environment E. The observation of this factor makes it possible to conclude that the person is not apathetic, due to the change in emotional state thereof following the emission of a particular state from the environment thereof.

Advantageously, the determination method 100 is periodically renewed to monitor at various successive times tₖ over time, during a period [tᵢ,t_{f}], the change in the emotional contagion of the environment E to the person P during the period [tᵢ,t_{f}].

In one embodiment, the first signals Spᵢ(t) are continuously captured during the period [tᵢ,t_{f}]. For an implementation of the method 100 for determining at a time tₖ included in the period [tᵢ,t_{f}], only the values acquired from these first signals Spᵢ(t) during the period [tᵢ,tₖ] are used during steps E200a, E200b and E300.

Similarly, in this embodiment, the second signals Seⱼ(t) are continuously captured during the period [tᵢ,t_{f}]. For an implementation of the method 100 for determining at a time tₖ included in the period [tᵢ,t_{f}], only the values acquired from these second signals Ssⱼ(t) during the period [tᵢ,tₖ] are used during steps E200a, E200b and E300;

In another embodiment, only the values of the first signals Spᵢ(t) and the second signals Ssⱼ(t) included within a window [tₑ−T,tₑ] of duration T are taken into account during the steps E200a, E200b and E300. For example, the duration T is 7 days.

If the duration T corresponds to the period of time between two determinations of emotional signatures of the person P and between two determinations of signatures of the environment E, the degree of similarity determined in step E300 is an instantaneous degree of similarity.

Advantageously, before a first evaluation time tₑ, a calibration period takes place during which the determination method 100 is implemented a plurality of times in order to determine a degree of similarity typical of the person P. Thus, from the first evaluation time tₑ, the degree of similarity determined in step 300 of the determination method 100 will be compared with the degree of similarity typical of the person P, so as to be able to estimate whether the state of health of the person P is good relative to the usual state of health thereof, or whether the person P tends towards a negative state.

One of the advantages of the invention lies in the fact that the determination method is carried out based on signals captured objectively. The determination method has a high reproducibility due to this objective evaluation, unlike clinical studies performed on the basis of questionnaires filled out subjectively either by qualified people or by subjects analysed, or by operators having more or less experience.

A second aspect of the invention relates to the determination system 1.

The determination system 1 is configured to implement the method, previously described, for determining an emotional state of a person P, to monitor the state of health thereof.

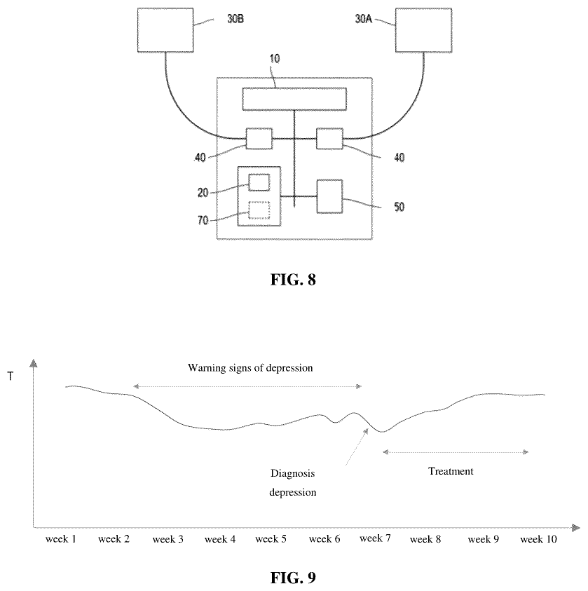

FIG. 8 is a schematic diagram showing some of the components of an example of the determination system 1.

The determination system 1 may be implemented as a single hardware device, for example in the form of a desktop personal computer (PC), a laptop, a personal digital assistant (PDA), a smartphone, a server, a console or may be implemented on separate hardware devices interconnected by one or more communication links, with wired and/or wireless segments. The determination system 1 may for example be in communication with one or more cloud computing systems (i.e. in the cloud), one or more remote servers or devices to implement the functions described in the present document for the device concerned. The determination system 1 may also be implemented itself as a cloud computing system.

As schematically represented in FIG. 8, the determination system 1 can comprise at least one processor 10 configured to access at least one memory 20 comprising a computer program code 70. The computer program code may comprise instructions configured to lead the determination system 1 to execute one or more or all of the steps of the determination method 100 described above.

The processor 10 can thus be configured to store, read, load, interpret, execute, and/or otherwise process the computer program code 70 stored in the memory 20 so that, when the instructions encoded in the computer program code are executed by the at least one processor 10, the system 1 executes one or more steps of the determination method 100 described here.

The processor 10 may be any suitable microprocessor, microcontroller, integrated circuit, central processing unit (CPU), graphics processing unit (GPU), or tensor processing unit (TPU), comprising at least one hardware-based processing processor or core.

The memory 20 may comprise a random access memory (RAM), a cache, a non-volatile memory, a backup memory (for example, programmable or flash memories), a read-only memory (ROM), a hard drive (HDD), a solid state drive (SSD), or any combination thereof. The ROM of the memory 20 may be configured to store, among other things, an operating system of the system S and/or one or more computer program codes of one or more software applications. The RAM of the memory 20 may be used by the processor 10 for the temporary storage of data.

The system 1 may further comprise one or more communication interfaces 40 (for example, network interfaces for accessing a wired/wireless network, including an Ethernet interface, a WIFI interface, USB interfaces, etc. The determination system 1 may comprise other associated hardware such as user interfaces such as a 2D visual rendering interface 30A, in our case containing here a 3D screen and 3D goggles, or haptic interfaces 30B, or any other interfaces (for example keyboard, mouse, display screen, etc.) connected via one or more suitable communication interfaces 40 with the processor. The determination system 1 may also comprise a media reader 50 for reading a computer-readable external storage medium. The processor 10 is connected to each of the other components in order to control the operation thereof.

A third aspect of the invention relates to a computer program product including instructions for implementing the steps of the method for determining, at an evaluation time, an emotional state of a subject immersed in an environment for the purpose of monitoring a state of health of said subject during the execution of the program by a processor of a programmable device. For example, the instructions are part of the computer program code 70 stored in the memory 20 of the determination system 1.

In particular, the computer program product implements the steps E200a, E200b, E300.

### EXAMPLES

The present invention will be better understood upon reading the following examples which illustrate the invention without limitation.

### Example 1

In this example, the person P is an elderly person in the bedroom thereof whose state of health is to be monitored based on the emotional state thereof. The person P is facing the television thereof.

The environment E mainly includes the programme broadcast on the television.

The determination system 1 comprises a small robot comprising a webcam and connected to the Internet.

The sensor C₁ therefore corresponds to the image sensor of the webcam, directed towards the face of the person P.

The environment sensor CE₁ is the webcam microphone that records sound coming from the programme broadcast on the television.

The small robot comprises an onboard system with a programmable device that can analyse the images recorded by the sensor C₁, in other words that can perform step E200a of the determination method 100. Typically, analysing the facial expressions of the elderly person P in the recorded images makes it possible to determine the corresponding emotional signatures. A method such as that described in “Estimation of continuous valence and arousal levels from faces in naturalistic conditions”, Toisoul et al., Nature Machine Intelligence volume 3 (2021) may be used.

The programmable device also analyses the sound recorded by the environment sensor CE₁, in other words, it performs step E200b of the determination method 100. Typically, the analysis of the recorded sound makes it possible to determine the corresponding signatures of the environment. A method such as that described in “Dawn of the transformer era in speech emotion recognition: closing the valence gap”, Wagner (2022) may be used.

These in-situ analyses make it possible to ensure data confidentiality since the images are never stored in a physical memory but are processed in real time in the acquisition stream.

Following steps E200a and E200b, the small robot sends the emotional signatures of the person P and the signatures of the environment E.

The step E300 of determining the degree of similarity is performed in the cloud and the degree of similarity determined can be received by healthcare professionals according to the elderly person P.

### Example 2

This example illustrates a use of the determination method 1 and of the determination system 1 previously described in the context of monitoring and taking care of a person P arriving in an RCFE.

It is assumed that the person P is monitored by a team of healthcare professionals and that the determination method 100 is regularly implemented with a determination system 1.

The team of healthcare professionals monitors, at each iteration of the determination step E200a relating to the person P, the corresponding emotional tone based on all of the emotional signatures of the person P, also previously denoted first together. For example, and as explained above, emotional tone can be quantified by the total area occupied in the valence/arousal space by the first set. The higher the emotional tone, the healthier the person P is. The lower the emotional tone, the more the person P tends towards a negative state.

Thus, if a decrease in emotional tone is noted, the team of healthcare professionals can initiate a panel of care for the person P, while monitoring the change in the emotional tone thereof.

FIG. 9 illustrates the change in the emotional tone of the person P. It is observed that after one week the person P begins to become depressed. This is indeed a symptomatic event of entering the institution. Depression is diagnosed two weeks later based on the observation of warning signs of depression. If assistance is provided to the person P, such as advice to encourage them to connect with residents of the facility, an increase in the emotional tone of the person being followed may be observed from the time of the observation of warning signs and care, as seen in FIG. 9.

### REFERENCE SIGNS

tₑ: evaluation time
S(tₑ): emotional state of a person P at the time tₑ
CPi, i between 1 and I: sensors
E: environment
CEj, j between 1 and J: environment sensors
Spi(t), between 1 and I: first signals
Sej(t), j between 1 and J: second signals
R: space representative of the emotions
M1: heat map of emotions
M2: heat map of states of the environment

1-14. (canceled)

15. A system for determining, at an evaluation time, an emotional state of a person immersed in an environment for the purpose of monitoring an emotional state of said person, comprising at least one sensor, at least one environment sensor, and a programmable device, said programmable device being configured to:
a. receive, during all or part of a time period preceding the evaluation time, at least one first signal acquired by the at least one sensor, said first signal being representative of a first time sequence of behavioural and/or physiological attributes of the person during said time period;
b. receive, during all or part of said time period, at least one second signal acquired by the at least one environment sensor, said second signal being representative of a second time sequence of a state of the environment during said time period;
c. determine, based on the at least one first signal, a third time sequence of emotional signatures of the person during said time period, said third sequence forming a first set of emotional signatures of the person, each of the emotional signatures of the person of the third sequence being represented by a position in a space representative of the emotions;
d. determine, based on the at least one current second signal, a fourth time sequence of signatures of the environment during said time period, said fourth sequence forming a second set of signatures of the environment, each of the signatures of the environment being represented by a position in said space representative of the emotions;
e. determine a degree of similarity between the first set of emotional signatures of the person and the second set of signatures of the environment,
f. determine the emotional state of the person at the evaluation time from the determined degree of similarity.

16. The determination system according to claim 15, wherein the at least one second signal being acquired by the at least one environment sensor chosen from: a camera, a webcam, a three-dimensional camera, a microphone, a means for accessing a remote server storing information relating to one or more audiovisual, visual, or audio content broadcast during all or part of the time period, an electromagnetic detector, and said programmable device being configured to extract from the at least second signal the state of the environment, so as to obtain values representative of a state chosen from: an amount of movement in audiovisual or visual content, broadcast during all or part of said time period, a sound level and/or a frequency spectrum of an audiovisual or audio content broadcast during all or part of said time period, a sound level and/or a sound frequency spectrum in a neighbourhood close to the subject, an electromagnetic signal representative of a variation of an electromagnetic signature of a neighbourhood close to the subject.

17. The determination system according to claim 15, wherein said programmable device is configured to determine the degree of similarity between the first set of emotional signatures of the person and the second set of signatures of the environment via the calculation of a distance in said space representative of the emotions between the first set of emotional signatures of the person and the second set of signatures of the environment.

18. The determination system according to claim 17, wherein said distance is calculated by means of a cosine similarity between a first vector representative of the first set of emotional signatures of the person and the second set of signatures of the environment.

19. The determination according to claim 17, wherein the calculation of said distance comprises a weighting dependent on the positions of the emotional signatures of the person of the first set and of the signatures of the environment of the second set in said space representative of the emotions.

20. The determination system according to claim 17, wherein the calculation of said distance comprises a time-dependent weighting corresponding to the emotional signatures of the person in the third time sequence and to the signatures of the environment in the fourth time sequence.

21. The determination system according to claim 19, wherein the calculation of said distance is carried out between the emotional signatures of the person of a first sub-sequence of the third time sequence and the signatures of the environment of a second sub-sequence of the fourth time sequence, said first sub-sequence and second sub-sequence corresponding to a time sub-sequence of said time period.

22. The determination system according to claim 15, wherein:
the at least one first signal is acquired by the at least one sensor chosen from: a camera, a webcam, a three-dimensional camera, a microphone, an inertial unit, a physiological sensor, a headset dedicated to electroencephalographic measurements,
and in that said programmable device is configured to extract from the at least one first signal the behavioural and/or physiological attributes in the first time sequence so as to obtain values representative of at least one of the following attributes: a gesture of the person, a movement of the person, a facial expression of the person, a movement of the eyes of the person, physiological parameters of the person, an interaction with an electronic device.

23. A computer-implemented method for determining, at an evaluation time, an emotional state of a person immersed in an environment for the purpose of monitoring an emotional state of said person, said method comprising the following steps of:
g. receiving, during all or part of a time period preceding the evaluation time, at least one first signal acquired by the at least one sensor, said first signal being representative of a first time sequence of behavioural and/or physiological attributes of the person during said time period;
h. receiving, during all or part of said time period, at least one second signal acquired by the at least one environment sensor, said second signal being representative of a second time sequence of a state of the environment during said time period;
i. determining, based on the at least one first signal, a third time sequence of emotional signatures of the person during said time period, said third sequence forming a first set of emotional signatures of the person, each of the emotional signatures of the person of the third sequence being represented by a position in a space representative of the emotions;
j. determining, based on the at least one current second signal, a fourth time sequence of signatures of the environment during said time period, said fourth sequence forming a second set of signatures of the environment, each of the signatures of the environment being represented by a position in said space representative of the emotions;
k. determining a degree of similarity between the first set of emotional signatures of the person and the second set of signatures of the environment,
l. determining the emotional state of the person at the evaluation time from the determined degree of similarity.

24. The method according to claim 23, wherein determining the degree of similarity between the first set of emotional signatures of the person and the second set of signatures of the environment comprises calculating a distance in said space representative of the emotions between the first set of emotional signatures of the person and the second set of signatures of the environment.

25. The method according to claim 24, wherein said distance is calculated by means of a cosine similarity between a first vector representative of the first set of emotional signatures of the person and the second set of signatures of the environment.

26. The method according to claim 24, wherein the calculation of said distance comprises a weighting dependent on the positions of the emotional signatures of the person of the first set and of the signatures of the environment of the second set in said space representative of the emotions.

27. The method according to claim 24, wherein the calculation of said distance comprises a time-dependent weighting corresponding to the emotional signatures of the person in the third time sequence and to the signatures of the environment in the fourth time sequence.

28. A non-transitory computer-readable storage medium comprising instructions which, when executed by a computer, cause the computer to carry out the method according to claim 23.
